(19) United States
(12) Patent Application Publication
(10) Pub. No.: US 2016/0095908 A1
(43) Pub. Date: Apr. 7, 2016

(54) Botulinum Toxin for Primary Disorders of Mood and Affect using Neurotransmitter CNS imaging Studies

(71) Applicant: Botulinum Toxin Research Associates, Inc., Quincy, MA (US)

(72) Inventors: Gary E. Borodic, Quincy, MA (US); Martin Acquadro, Wellesley, MA (US)

(21) Appl. No.: 14/966,759

(22) Filed: Dec. 11, 2015

Related U.S. Application Data
(63) Continuation-in-part of application No. 14/464,740, filed on Aug. 21, 2014.
Application No. 14/464,740 is a division of application No. 11/447,984, filed on Jun. 7, 2006, now Pat. No. 8,926,991.
(60) Provisional application No. 60/690,162, filed on Jun. 14, 2005.
(60) Provisional application No. 60/693,771, filed on Jun. 27, 2005.
(60) Provisional application No. 60/721,060, filed on Sep. 28, 2005.
(60) Provisional application No. 60/738,981, filed on Nov. 23, 2005.

(51) Int. Cl. A61K 38/48; A61K 9/00

(52) U.S. Cl. CPC A61K 38/4893; A61K 9/0085; C12Y 304/24069

(57) ABSTRACT

The invention provides methods for treating primary disorders of mood and affect, including depressive disorders, anxiety, and sleep disorders and CNS disorders comprising the administration of a neurotoxin.

This application claims benefit to U.S. patent application Ser. No. 14/464,740, filed on Aug. 21, 2014, which claims priority to U.S. patent application Ser. No. 11/447,984, filed on Jun. 7, 2006, which claims priority to four U.S. Provisional Application No. 60/690,162, filed Jun. 14, 2005; U.S. Provisional Application No. 60/693,771, filed on Jun. 27, 2005; U.S. Provisional Application No. 60/721,060, filed on Sep. 28, 2005; U.S. Provisional Application No. 60/738,981, filed on Nov. 23, 2005, all of which are hereby incorporated by reference in their entireties.

### FIELD OF THE INVENTION

The present invention relates to the treatment of primary disorders of mood and affect with a neurotoxin, including depressive, anxiety and sleep disorders as well as other CNS disorders. This invention deals with the use of neurotransmitter imaging of sections of the brain and central nervous system to select patients for botulinum administration and to select appropriate anatomic sites in the extracranial region in close proximity to the targeted brain and CNS area with abnormal neurotransmitter expression. The method allows for diffusion of botulinum toxin across the blood brain and anatomic surface brain barriers to safely and effectively deliver the botulinum against to the central nervous system, to effect a block in neurotransmitter function.

### BACKGROUND OF THE INVENTION

Depression is one of the most prevalent and pervasive forms of mental illness that affects individuals across age and gender lines. (Gainotti et al. (2001) J. Neural Neurosurg. Psychiatr. 71: 258-261; Wong et al. (2001) Nature Rev. Neurosci. 2: 343-351; Nestler et al. (2002) Neuron 34: 13-25). The lifetime risk of major depression is about 12% in men and about 25% in women, generally, (Kessler et al. (1994) Arch. Gen. Psychiatry 51: 8). In addition, about 5 to 10% of all patients in the primary care environment, present with major depression, whereas about 3 to 5% of patients are diagnosed with dysthymia. (Barrett et al. (1988) Arch. Gen. Psychiatry 45: 1100). In an in-patient setting, however, between 10 and 14% of all patients are diagnosed with major depression. (Blackburn et al. (1997) Br. J. Psychiatry 171: 328). Major depression is a particularly disabling and pernicious, in part, because it is recurring. The rate of relapse for patients with major depression is about 40% over a two-year period after a first episode. The occurrence of relapse increases to about 75% within a five year period after the diagnosis of a second episode of major depression. (Solomon et al. (2000) Am. J. Psychiatry 157: 229).

Depressive disorders are most commonly treated with three main classes of compounds: 1) monamine oxidase inhibitors; 2) heterocyclic antidepressants; and 3) selective serotonin reuptake inhibitors (SSRIs). The known and currently prescribed antidepressants are by numerous side effects. Monoamine oxidase inhibitors were the first class of antidepressants used clinically. Monoamine oxidase inhibitors, including isocarboxazid, pheneizine, and tranylcypromine, inhibit the metabolism of phenylethylamine and catabolism of dopamine, serotonin and norepinephrine. As a consequence of numerous dietary restrictions associated with the use of monoamine oxidase inhibitors, extensive side effects, including hypertension, headache, myoclonic jerk, sleep disruption, and gastrointestinal complications, monoamine oxidase inhibitors are currently not used as a first-line antidepressant. The tricyclic antidepressants, including, imipramine, desipramine, nortrypline, amitrypline, doxepin and protrypline, produce a variety of anticholinergic side effects, drowsiness, orthostatic hypotension, cardiac arrhythmias and weight gain. Although generally milder than the monoamine oxidase inhibitors and the tricyclic antidepressants, SSRIs also produce numerous side effects. For example, SSRIs, including fluoxetine, paroxetine, fluvoxamine, sertraline, and citalopram, are associated with gastrointestinal distress, jitteriness, agitation and sleep disruption.

In addition to the numerous side effects associated with traditional antidepressant medications, these therapeutics are also characterized by marginal efficacy. Several studies on the efficacy of antidepressant therapy for major depression have concluded that the treatment of acute disease or maintenance therapy is associated with a 50-60% response rate. (Schulberg et al. (1998) Arch. Gen. Psychiatry 55: 1121). The average absolute response rate between antidepressants and placebo is about 20-25%. (Williams et al. (2000) Ann. Intern. Med. 132: 743). Consequently, there is a current need for new antidepressant therapies.

In view of the sometimes severe adverse side effects and marginal efficacy of numerous antidepressant therapies, there is a great need for improved pharmaceuticals that effectively treat depressive disorders and sleep disorders without producing the side effects associated with treatments of depression and/or sleep disorders. The present invention provides compositions comprising botulinum toxin neurotoxin for the treatment of depressive and/or sleep disorders as well as other CNS disorders.

The effects of botulinum toxin-based pharmaceuticals for medicinal applications has traditionally been though to act on the peripheral motor and possibly sensory nerves. Such actions of these agents have been used to explain most of the beneficial effects for various indications including movement disorders, pain syndromes, autonomic based syndromes and spastic disorders. To date, clinical observations have been made by the inventor which are indicative of the involvement of the central nervous system and cannot be explained by peripheral effects. Effects on the central nervous system are observed even when botulinum toxin is administered to the scalp, facial or neck regions, including administration by any form of injection except intracranial injection. Such observations include: improvement in photophobia with peri-ocular injections; improvement in sleep patterns and relief of insomnia; improvement of anxiety out of proportion to problems corrected by physical/muscular impairments; improvement in depression out of proportion to problems created by physical/muscular impairment; and effects on dysmenorrheal symptoms and potential effects on gonadotropin hormones or other pituitary hormones.

Additionally, botulinum toxin has been shown to have effects on neurotransmission within the central nervous system when the agent is directly injected into brain parenchyma. Alterations have included depression in electrode depolarization, depression in glutamate release, GABA staining, and cleavage of SNAP-25 in duration consistent with botulinum toxin effect. Intraparenchymal brain injections have been associated with depression of seizure activity within the cerebral hemispheres when seizure provoking scarring is induced by caustic chemical injections. Direct injection into the brain is not practical and in fact unlikely to be conventionally practiced by a physician skilled in the treatment of seizure disorders because of the possibility and risk associated with induced hemorrhage, scarring, neuronal loss and placement difficulty, infection (meningitis) and inconvenience associated with necessary delivery mechanisms. Direct injection into the CNS is highly impractical because of such complications associated with invasive intracranial procedures. Described herein is a system for delivery to the central nervous system (through methods of administration and injection that expressly do not include transcranial, intrathecal or intraspinal injection) of botulinum toxin-based pharmaceuticals, allowing penetration into the central nervous system with enhanced convenience and safety, with fewer or mitigated adverse effects associated with direct delivery.

The present inventors have surprisingly and unexpectedly discovered criteria for the selection of subjects for the treatment of pain syndromes with botulinum toxin. The present invention provides methods for identifying subjects with an increased responsiveness to the treatment of pain with botulinum toxin. Specifically, the inventors have discovered that atopic disease is associated with various pain syndromes, and the presence of atopic disease and relief of pain by tactile stimulation, geste antagoniste phenomenon, seem to have predictive value in forecasting pain response to botulinum toxin.

The application of botulinum toxin for the treatment of myofacial pain initially included tension headaches, bruxism, temporal mandibular joint syndrome, lower-back pain, and post-surgical pain after cervical surgical incisions for the treatment of acoustic neuroma (posterior fossa brain tumor). Application of botulinum toxin for the treatment of migraine headaches became popular after the coincident observation that migraine headaches were relieved after the of botulinum toxin to efface facial wrinkles on the forehead.

Multiple case reports suggest that botulinum toxin is effective for the treatment of tension and migraine headaches, as well as forms of myofacial pain syndrome. Despite this suggestion, controlled trials using small numbers of patients in the study groups, have failed to demonstrate the efficacy of botulinum toxin for the treatment of myofascial and other forms of pain. (Wheeler et al. (1998) A randomized, double-blind, prospective pilot study of botulinum toxin injection for refractory, unilateral, cervicothoracic, paraspinal, myofascial pain syndrome. Spine 23(15): 1662-6). The ineffectiveness of botulinum toxin to treat a variety of pain syndromes, in controlled trial, has been attributed to small sample size and relatively low statistical power. The need for larger numbers of patients and further multi-center investigations have been deemed necessary to provide stronger evidence of effectiveness.

In view of case reports suggesting that botulinum toxin is indeed effective for the treatment of migraine-headache-pain syndromes, efforts were made to conduct larger-scale studies. In an initial multi-center controlled study sponsored by the Allergan Pharmaceutical Company, one of the largest suppliers of botulinum toxin A (BOTOX™), efficacy of botulinum toxin to prevent the repetitive occurrence of common migraine headaches (as defined by the International Headache Classification—1988) was suggested. The statistical significance of these results, however, was uncertain, inconsistent between treatment groups, and exhibited unexplained inverted dose response curves. (Silberstein et al. (2000) Botulinum toxin type A as a migraine preventive treatment. Headache 40(6): 445-50).

Migraine, tension headaches, myofascial pain of the head, and chronic atypical facial headaches are representative of primary-headache disorders (headaches not associated with structural pathology within the head or not secondary to another disease process). Treatment of these conditions is associated with very high placebo response rates (up to 35%), requiring large numbers of patients to detect significant differences in clinical trials between study and control groups. Utilization of selection criteria (study-induction criteria) that identify a more responsive patient population increases the response rate for subjects within treatment groups of controlled studies, which, in turn, allows a smaller test sample to establish therapeutic efficacy in controlled trials. More importantly, selection criteria (diagnostic criteria) are the basis for accurate and effective medical therapy for any condition. Parameters which identify patients more likely to respond to a given treatment allow: 1) prioritization among therapies when multiple therapeutic options exist; 2) avoidance of therapy unlikely to be successful; and 3) facilitation of informed consent from patients considering risks and benefit ratios. Effective selection criteria assist researchers to further understand mechanisms of action based on clinical evidence.

The present invention provides methods of selecting patients suffering from various pain syndromes, including, but not limited to, myofascial pain, muscle tension headache, and chronic post operative wound syndromes, based on retrospective and prospective analysis in the application of botulinum toxin for the treatment of pain syndromes involving the head and neck.

### SUMMARY OF THE INVENTION

The present invention provides methods of treating depressive, anxiety and sleep disorders comprising the administration of pharmaceutical compositions comprising neurotoxins.

The present invention provides methods for treating depression comprising the steps of: a) identifying a subject with a depressive disorder or identifying a subject with one or more symptoms of a depressive disorder; and b) administering an effective amount of a composition comprising a botulinum toxin and a pharmaceutically acceptable carrier to said subject.

The present invention also provides methods of treating anxiety comprising the steps of: a) identifying a subject with an anxiety disorder or identifying a subject with at least one symptom of an anxiety disorder; and b) administering an effective amount of a composition comprising a botulinum toxin and a pharmaceutically acceptable carrier to said subject.

The present invention also provides methods of treating sleep disorders comprising the steps of: a) identifying a subject with a sleep disorder or identifying a subject exhibiting at least one symptom of a sleep disorder; and b) administering an effective amount of a composition comprising a botulinum toxin and a pharmaceutically acceptable carrier to said subject.

The present invention also provides methods of treating circadian rhythm disorders comprising the steps of: a) identifying a subject with a circadian rhythm disorder; and b) administering an effective amount of a composition comprising a botulinum toxin and a pharmaceutically acceptable carrier to said subject.

The present invention also provides methods of delivering botulinum toxin across a blood-brain barrier comprising the steps of: a) identifying a subject with at least one neuropsychiatric disorder; and b) administering a composition comprising a neurotoxin and a pharmaceutically acceptable carrier to said subject in an amount sufficient to deliver said neurotoxin across the blood-brain barrier.

The present invention also provides methods of delivering botulinum toxin across a blood-brain barrier comprising the steps of: a) identifying a subject with at least one neuropsychiatric disorder; and b) administering a composition comprising a neurotoxin and a pharmaceutically acceptable carrier to said subject in an amount sufficient to deliver said neurotoxin across the blood-brain barrier, wherein said administration of said injection of neurotoxin blocks at least one neurotransmitter. In a preferred embodiment, the neurotransmitter is acetylcholine.

The present invention also provides methods of treating an anxiety disorder comprising the steps of: a) identifying a subject with at least one anxiety disorder or identifying a subject with one or more symptoms of an anxiety disorder; and b) administering to said subject a composition comprising a neurotoxin and a pharmaceutically acceptable carrier said composition is delivered across the blood-brain barrier in an amount sufficient to decrease cholinergic neuron transmission.

The present invention also provides methods of treating a sleep disorder comprising the steps of: a) identifying a subject with at least one sleep disorder or identifying a subject with one or more symptoms of a sleep disorder; and b) administering to said subject a composition comprising a neurotoxin and a pharmaceutically acceptable carrier said composition is delivered across the blood-brain barrier in an amount sufficient to decrease cholinergic neuron transmission. In a preferred embodiment, the composition decreases choline acetyltransferase activity. In another preferred embodiment, the composition decreases the synthesis of acetylcholine. In another preferred embodiment, the sleep disorder is insomnia. In another preferred embodiment, the sleep disorder is narcolepsy, restless leg syndrome or sleep apnea.

The present invention also provides methods of treating a circadian rhythm disorder comprising the steps of: a) identifying a subject with at least one circadian rhythm disorder or identifying a subject with one or more symptoms of a circadian rhythm disorder; and b) administering to said subject a composition comprising a neurotoxin and a pharmaceutically acceptable carrier said composition is delivered across the blood-brain barrier in an amount sufficient to decrease cholinergic neuron transmission. In a preferred embodiment, the composition decreases choline acetyltransferase activity. In another preferred embodiment, the composition decreases the synthesis of acetylcholine.

The present invention also provides methods of treating a depressive disorder comprising the steps of: a) identifying a subject with at least one depressive disorder or identifying a subject with one or more symptoms of a depressive disorder; and b) administering to said subject a composition comprising a neurotoxin and a pharmaceutically acceptable carrier said composition is delivered across the blood-brain barrier in an amount sufficient to decrease cholinergic neuron transmission. In a preferred embodiment, the composition decreases choline acetyltransferase activity. In another preferred embodiment, the composition decreases the synthesis of acetylcholine.

The present invention provides methods of selecting a subject for the treatment of pain with botulinum toxin, comprising the step of identifying a subject suffering from a pain syndrome and a condition selected from the group consisting of a depressive disorder, an anxiety disorder and a sleep disorder, wherein the identification of a subject with a pain syndrome and a condition selected from the group consisting of a depressive disorder, an anxiety disorder and a sleep disorder is predictive of increased responsiveness to the treatment of pain with botulinum toxin. In a preferred embodiment, the pain syndrome is any one or a combination of the pain syndromes selected from the group consisting of: myofacial pain; migraine headache; post operative would pain; sinusitis-related headaches; muscle tension headaches; post-traumatic headaches; cluster headaches; temporal mandibular joint syndrome; fibromyalgia; atypical facial pain; post incisional wound pain; cervical radiculopathy; and whiplash.

In another embodiment of the present invention, subjects suffering from a condition selected from the group consisting of a depressive disorder, an anxiety disorder and a sleep disorder were identified by determining that a subject has a medical history of a depressive disorder, an anxiety disorder, or a sleep disorder, respectively.

The present invention also provides methods of identifying a subject with increased responsiveness to treating a pain disorder with botulinum toxin, comprising the step of screening a population of subjects to identify those subjects that suffer from a pain disorder and a condition selected from the group consisting of a depressive disorder, an anxiety disorder and a sleep disorder, wherein the identification of a subject with a pain syndrome and a condition selected from the group consisting of a depressive disorder, an anxiety disorder and a sleep disorder is predictive of increased responsiveness to the treatment of pain with botulinum toxin. In a preferred embodiment, the pain syndrome is any one or a combination of the pain syndromes selected from the group consisting of: myofacial pain; migraine headache; post operative would pain; sinusitis-related headaches; muscle tension headaches; post-traumatic headaches; cluster headaches; temporal mandibular joint syndrome; fibromyalgia; atypical facial pain; post incisional wound pain; cervical radiculopathy; and whiplash.

The present invention provides a method that comprises the steps of identifying or diagnosing a pain syndrome; diagnosing or eliciting a history of a condition selected from the group consisting of a depressive disorder, an anxiety disorder and a sleep disorder; and classifying the identified pain syndrome as one with increased responsiveness to treatment with botulinum toxin. In one embodiment, a pain syndrome is identified according to the International Headache Classification System (The International Headache Society (I.H.S.)).

The present invention provides a method of selecting patients for the treatment of human headache disorders with a botulinum toxin based pharmaceutical, comprising diagnosing headache type occurring in a patient suffering from a depressive disorder, an anxiety disorder, obsessive compulsive behavioral traits, or a sleep disorder and administering a therapeutically effective amount of botulinum toxin. In one embodiment the headache disorder is migraine, tension headache, combined tension and migraine headache, myofascial headache, sinus headache, headache associated with temporal mandibular joint syndrome, headache associated with fibromyalgia, or headache associated with neuralgia.

The present invention also provides a method of selecting patients for the treatment of human facial pain disorders with a botulinum toxin based pharmaceutical, comprising diagnosing a facial pain disorder occurring in a patient suffering from a depressive disorder, an anxiety disorder, obsessive compulsive behavioral traits, or a sleep disorder and administering a therapeutically effective amount of botulinum toxin. In one embodiment, the facial pain disorder is trigeminal neuralgia, the facial pain disorder is associated with bruxism, or the facial pain disorder is post operative chronic surgical wound pain.

The compositions of the present invention comprise botulinum toxin and a pharmaceutically acceptable carrier. In a preferred embodiment, the botulinum toxin is immunotype A, B, C, D, E, F, or G. In a more preferred embodiment, the botulinum toxin is botulinum toxin type A from Hall strain Clostridium botulinum.

The methods of the present invention may preferably be practiced by administering the botulinum toxin compositions by injection, including transcutaneous, percutaneous, subcutaneous, intraperitoneal, transdermal, intramuscular and intraosseous, but expressly not intracranial, transcranial, intrathecal or intraspinal injection or administration. In one embodiment, there are at least two injection sites. In another embodiment, the injections are multifocal. The botulinum toxin may be preferably administered to the forehead, scalp or neck or other locations such as the periocular region and other areas of the face that enhance maximize venous drainage from the site of administration to the central nervous system (CNS). In another embodiment, the botulinum toxin may be administered to the soft tissues outside the neurocranium. In another embodiment, the botulinum toxin may be administered in locations that maximize uptake by the portal hypophyseal drainage.

Examples of compounds and formulations which can be used in the present invention include botulinum toxin stabilized with a protein such as serum albumin or hyaluronidase. In a preferred embodiment the serum albumin or hyaluronidase is recombinant. In another embodiment, the serum albumin is present at a concentration of greater than 500 .mu.g/100 LD.sub.50 units botulinum toxin. The botulinum toxin pharmaceutical may be further stabilized with a simple stabilizing sugar or polysaccharide (e.g. sucrose, lactose or trehalose). The botulinum toxin is preferably a monocomponent neurotoxin of a molecular weight of 150,000 daltons that is free of complex botulinum toxin proteins. The compositions disclosed herein may also comprise a polyethylene glycol polymer; a vegetable fat-based nanoemulsion; and any nanoemulsion using one or more monounsaturated or polyunsaturated oils. In a preferred embodiment, the botulinum toxin compositions used in the methods of the present invention are formulated to enhance penetration of the botulinum toxin into and through the skin.

Recent advances in pharmaceutical technology have focused on enhanced delivery systems such as transdermal or transcutaneous delivery systems. Such systems are thought to be more convenient and associated with less pain. The problems associated with such systems include poor penetration of materials through the epidermis and dermis. Hyaluronidase offers improved penetration.

A pharmaceutical composition comprising botulinum neurotoxin, hyaluronidase, and sugars (both simple and oligosaccharides) is suitable for the methods of the present invention. The botulinum toxin pharmaceutical formulations suitable for use in for the methods of the present invention are preferably devoid of any human blood or recombinant blood products and will be either stabilized in flash or freeze dried form. The pH is preferably between pH 3.0 to 7.4 and the preparation may be used as an injection, transdermal or topical agent. The botulinum toxin pharmaceutical formulations suitable for use in for the methods of the present invention may be administered by injection, needleless delivery systems and methods requiring disruption techniques such as electroporation, sonication, and high pressure air gas flow injection or in the form of a micro-needle. Micro-needles are generally from 150 to 600 microns. Furthermore, the botulinum toxin pharmaceutical formulations suitable for use in for the methods of the present invention may further comprise polycationic proteins.

Currently, hyaluronidase is available at a number of specific activities. For example, sheep based materials can have a specific activity of 1,500 Upper mg or 1.5 Upper mcg. Typically, 75-300 U are used for injection, such as conducted with peri-bulbar anesthesia for intra-ocular surgery. This would correspond to about 100-450 mg in mass of enzymatic protein, enough to act as a stabilizing excipient.

Prior studies have shown that a protein excipient, such as human serum albumin, can stabilize the botulinum toxin. Test studies conducted demonstrate that hyaluronidase also stabilizes the botulinum toxin at the same levels observed for the human serum albumin.

The compositions disclosed herein may be such that the doses are formulated into a concentration suitable for administration as an eye drop to facilitate transconjunctival penetration for the treatment of ocular surface diseases. The compositions disclosed herein may be such that the LD.sub.50 units range from 1.25 U-3,000 units of botulinum toxin type A. The compositions disclosed herein may be such that the LD.sub.50 units range from 1.25-20,000 U of botulinum type B. The compositions disclosed herein may be such that the formulation is delivered into the nose or oral cavity as an aerosol to facilitate intracranial delivery of a botulinum toxin based pharmaceutical. The compositions disclosed herein may be such that the formulation is delivered into the ear canal as an aerosol to facilitate intracranial delivery of a botulinum based pharmaceutical

The present invention provides methods for delivering a botulinum toxin based pharmaceutical to the central nervous system of a subject by any injection or topical application method, except intracranial, transcranial, intrathecal or intraspinal injection, in a therapeutically effective amount sufficient to decrease at least one central nervous system neurotransmitter when compared to an untreated subject. In a preferred embodiment, the at least one central nervous system neurotransmitter is glutamate, norepinephrine, or acetyl-choline. In a more preferred embodiment, the at least one central nervous system neurotransmitter is glutamate. In another embodiment, the methods of the present invention decrease at least one central nervous system neurotransmitter when compared to an untreated subject sufficiently to reduce at least one symptom of insomnia, a sleep disorder, an anxiety disorder, a depressive disorder, dysmenorrhea, an appetite or eating disorder, or a seizure disorder. In a preferred embodiment the seizure disorder is generalized, focal motor, or partial complex.

Glutamate is a neurotransmitter that exhibits endogenous neurotoxic activity that is observed in a number of neurodegenerative diseases and disorders, vascular accidents such as stroke and in seizure disorders. For example, subjects with mild to moderate dementia and probable Alzheimer's Disease have been shown to exhibit elevated levels of glutamate in the central nervous system. Elevated glutamate in the central nervous system is reflective of increased glutamatergic activity in the early stages of Alzheimer's Disease. The progressive neuronal loss observed in Alzheimer's Disease and other neurodegenerative disorders and diseases correlate with elevated glutamate and the increased excitotoxicity associated with elevated levels of this neurotransmitter.

The present invention provides methods for reducing glutamate levels in the central nervous system, the brain or portions of the brain comprising the step of administering a botulinum toxin pharmaceutical to a subject, by any injection or topical application method, except intracranial, transcranial, intrathecal or intraspinal injection, in an amount sufficient to reduce glutamate levels in the central nervous system, the brain or portions of the brain compared to an untreated subject.

The present invention provides methods for neuroprotection comprising the step of administering a botulinum toxin pharmaceutical to a subject, by any injection or topical application method, except intracranial, transcranial, intrathecal or intraspinal injection, in an amount sufficient to reduce neuronal loss in the central nervous system, the brain or portions of the brain compared to an untreated subject.

The present invention also provides methods for delivering a botulinum toxin based pharmaceutical to the central nervous system of a subject by injection into the nasal sinuses in a therapeutically effective amount sufficient to decrease at least one central nervous system neurotransmitter when compared to an untreated subject. In a preferred embodiment, the at least one central nervous system neurotransmitter is glutamate, norepinephrine, or acetyl-choline. In a more preferred embodiment, the at least one central nervous system neurotransmitter is glutamate. In another embodiment, the methods of the present invention decrease at least one central nervous system neurotransmitter when compared to an untreated subject sufficiently to reduce at least one symptom of insomnia, a sleep disorder, an anxiety disorder, a depressive disorder, dysmenorrhea, or a seizure disorder. In a preferred embodiment the seizure disorder is generalized, focal motor, or partial complex.

The present invention also provides methods for delivering a botulinum toxin based pharmaceutical to the central nervous system of a subject by any injection or topical application method, except intracranial, transcranial, intrathecal or intraspinal injection, in a therapeutically effective amount sufficient to decrease at least one central nervous system neurotransmitter when compared to an untreated subject. In a preferred embodiment, the at least one central nervous system neurotransmitter is glutamate, nor-epinephrine, or acetyl-choline. In a more preferred embodiment, the at least one central nervous system neurotransmitter is glutamate. In another embodiment, the methods of the present invention decrease at least one central nervous system neurotransmitter when compared to an untreated subject sufficiently to reduce an agitated behavior associated with mental retardation, schizophrenia, Huntington's Chorea or Alzheimer's Disease.

The present invention also provides methods for delivering a botulinum toxin based pharmaceutical to the central nervous system of a subject by any injection or topical application method, except intracranial, transcranial, intrathecal or intraspinal injection, in a therapeutically effective amount sufficient to decrease at least one central nervous system neurotransmitter when compared to an untreated subject. In a preferred embodiment, the at least one central nervous system neurotransmitter is glutamate, nor-epinephrine, or acetyl-choline. In a more preferred embodiment, the at least one central nervous system neurotransmitter is glutamate. In another embodiment, the methods of the present invention decrease at least one central nervous system neurotransmitter when compared to an untreated subject sufficiently to reduce at least one symptom of a neurodegenerative disease associated with inflammation.

The present invention also provides for the use of botulinum toxin or a botulinum toxin composition of the present invention in the production of a medicament for the treatment of any one of the disorders, diseases or conditions disclosed herein, including depressive disorders, anxiety disorders, sleep disorders, circadian rhythm disorders, neuropsychiatric disorders, Alzheimer's Disease and the like, and for the treatment of pain, such as various headache pain, associated with a pain syndrome.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 depicts the decreased glutamate receptor activity in the neostriatum of a botulinum-toxin-treated mouse as compared to an untreated mouse.

FIG. 2 shows graphic techniques locating glutamate deviations.

FIG. 3 shows areas over the frontal cortex and temporal cortex are shown to express excessive glutamate.

FIG. 4 shows that the regions of the brain are topically analyzed for areas of excessive GABA neurotransmission and extra cranial injection strategy is targeted to treat these areas.

### DETAILED DESCRIPTION OF THE INVENTION

### A. Definitions

As used herein, “administration” of a composition means any route of administration, including but not limited to oral, nasal, transcutaneous, percutaneous, subcutaneous, intraperitoneal, transdermal, intramuscular and intraosseous, but expressly excludes administered by any method, except intracranial, transcranial, intrathecal or intraspinal injection.

As used herein, “Botulinum toxin” means a protein toxin and its complexes isolated from strains of Clostridium botulinum, including various immunotypes such as A, B, C1, C2, C3, D, E, F and G.

As used herein, “depressive disorder” means major depression, dysthymia, and atypical depression or depression not otherwise specified.

As used herein, “an effective amount” is an amount sufficient to reduce one or more symptoms associated with a depressive, anxiety or sleep disorder or any of the disorders described herein.

As used herein, the term “pharmaceutically acceptable carrier” means a chemical composition with which the active ingredient may be combined and which, following the combination, can be used to administer the active ingredient to a subject. “Pharmaceutically acceptable carrier” also includes, but is not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

As used herein, “increased responsiveness” refers to an increase in the ratio of subjects responsive to pain treatment with botulinum toxin to total subjects (responsive and unresponsive to botulinum toxin).

As used herein, “response ratio” refers to the ratio of subjects responsive to pain treatment with botulinum toxin to total subjects (responsive and unresponsive to botulinum toxin).

As used herein, the term “screening a population” means a retrospective review and analysis of the medical history of a subject or an identification of a specific contemporaneous diagnosis.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, and other mammals.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient. In addition to the active ingredient, a pharmaceutical composition of the invention may further comprise one or more additional pharmaceutically active agents. Particularly contemplated additional agents include anti-emetics and scavengers such as cyanide and cyanate scavengers. Controlled- or sustained-release formulations of a pharmaceutical composition of the invention may be made using conventional technology.

As used herein, the term “physiologically acceptable” ester or salt means an ester or salt form of the active ingredient which is compatible with any other ingredients of the pharmaceutical composition, which is not deleterious to the subject to which the composition is to be administered.

### B. Depressive Disorders

Depressive disorders encompass the diagnoses of major depression, dysthymia, and atypical depression or depression not otherwise specified (“minor depression”). The different subgroups of depressive disorders are categorized and defined by the Diagnostic and Statistical Manual of Mental Disorders, Fourth Edition (DSM-IV). (American Psychiatric Association. Diagnostic and Statistical Manual of Mental Disorders, 4.sup.th Ed., Primary Care Version (DSM-IV-PC). American Psychiatric Association Press, Washington, D. C. 1995). According to the DSM-IV, a diagnosis of “major depression” requires that a patient present with at least five of the following nine symptoms during the diagnostic period: 1) depressed mood most of the day (most acute in the morning); 2) markedly diminished interest or pleasure in nearly all activities (anhedonia); 3) significant weight loss or gain; 4) insomnia or hypersomnia; 5) psychomotor agitation or retardation; 6) fatigue or energy loss; 7) feelings of guilt and worthlessness; 8) impaired concentration and indecisiveness; and 9) recurring thoughts of death or suicide. To support a diagnosis of major depression, a depressed mood or loss of interest (anhedonia) must be one of the five observed symptoms. In contrast, a diagnosis of “atypical depression” or “depression not otherwise specified” (also referred to as “minor depression”), the most common form of depression, requires between 2 and 4 depressive symptoms that are present daily or for most of the day for at least a two week period. Dysthymia is a chronic, low intensity mood disorder characterized by anhedonia, low self esteem and low energy that persists for more than two years, consecutively. Seasonal affective disorder is considered to be a form of major depression characterized by seasonal variation.

Depressive disorders do not include normal emotional reactions, a normal grief reaction or reactions secondary to an organic cause such as a physical illness or drug exposure. As used herein, depressive disorders refer to primary disorders of mood and sleep patterns and not secondary or reaction disorders. Such reactionary disorders occur secondarily to other medical disorders such as hyperhydrosis, cervical dystonia, migraine headache, tension headaches, various pain syndromes, jaw spasms, blepharospasm, strabismus, inflammatory local and systemic diseases, post operative pain syndromes, hemifacial spasms, cancer, myocardial infarction, stroke, degenerative neurological diseases, or any other physical ailment causing an emotional reaction.

### C. Anxiety

Anxiety is a group of disorders characterized by a number of both mental and physical symptoms, with no apparent explanation. Apprehension, fear of losing control, fear of going “crazy”, fear of pending death, impending danger, or uneasiness are among the most common mental symptoms. Common physical symptoms include dizziness, lightheadedness, chest pain, abdominal pain, nausea, increased hear rates or diarrhea. Chronic anxiety, also referred to as Generalized Anxiety Disorder, manifests as persistent worries, fears, and negative thoughts lasting a minimum of six months. Chronic anxiety often results in excessive worry over daily activities, headaches and nausea. Sleep disorders or early awakening, depression, tension, muscle aches and fatigue can all accompany chronic anxiety.

Acute anxiety, or Panic Disorder, comes on as a sudden attack or fear accompanied by symptoms that may resemble a heart attack, such as palpitations, chest pain and dizziness. Shortness of breath, stomach upset, chills, cold sweats, hot flashes, or irrational fears of death can combine with these symptoms to create a terrifying experience for the individual experiencing them. Excessive levels of nor epinephrine are seen to increase the rates of breathing and pulse in panic attack sufferers. Post-traumatic Stress Disorder is also classed as an anxiety disorder, and can be triggered by anyone experiencing or witnessing a deeply traumatic event. Some symptoms of Post-Traumatic Stress Disorder can be anger, depression, emotional numbness, flashbacks, nightmares and a tendency to startle easily.

Phobias, or irrational fears, and Obsessive Compulsive Disorder, a tendency towards repetitive or uncontrollable behavior, are also classed with anxiety disorders. These may co-exist together, as many individuals with obsessive compulsive disorder have phobias of germs or lack of cleanliness and may wash their hands or bathe excessively.

Anxiety disorders do not include normal emotional reactions, a normal reaction to stress or reactions secondary to an organic cause such as a physical illness or drug exposure.

### D. Sleep Disorders

Circadian rhythm describes the approximately 24-hour cycles that are generated by an organism. Most physiological systems demonstrate circadian variations. The systems with the most prominent variations are the sleep-wake cycle, thermoregulation, and the endocrine system. Circadian rhythm disturbances can be categorized into two main groups: transient disorders (e.g., jet lag, altered sleep schedule due to work, social responsibilities, and illness) and chronic disorders. The most common chronic disorders are delayed sleep-phase syndrome (DSPS), advanced sleep-phase syndrome (ASPS), and irregular sleep-wake cycle. Katzenberg et al. have suggested a genetic correlation (i.e., clock polymorphisms) to circadian rhythm patterns. DSPS is characterized by a persistent inability (more than 6 mo) to fall asleep and awaken at socially accepted times. Once asleep, these patients are able to maintain their sleep and have normal total sleep times. (In contrast, patients with insomnia have a lower than normal total sleep time, due to difficulties in initiating or maintaining sleep.) ASPS is characterized by persistent early evening sleep onset (between 6:00 and 9:00 pm) with an early morning wake-up time, generally between 3:00 and 5:00 am. ASPS occurs much less frequently than DSPS and is seen most commonly in the elderly and in individuals who are depressed.

The neural basis of the circadian rhythm, the suprachiasmatic nuclei (SCN), is located in the anterior ventral hypothalamus and has been identified as the substrate that generates circadian activity. Lesions of the SCN produce loss of circadian rhythmicity of the sleep-wake cycle, the activity-rest cycle, skin temperature, and corticosteroid secretion. Other pacemakers exist that are not located in the SCN. For instance, core body temperature rhythm persists in spite of bilateral ablation of SCN. Furthermore, free-running studies have provided evidence for multiple circadian oscillators. Under free-running conditions, circadian rhythm may split into independent components.

The SCN are the site of the master circadian clock in mammals. The SCN clock is mainly entrained by the light-dark cycle. Light information is conveyed from the retina to the SCN through direct, retinohypothalamic fibers. The SCN also receive other projections, like cholinergic fibers from basal forebrain. Cholinergic afferents and transmission have been shown to be involved in regulation of light-induced circadian rhythms. (Erhardt et al. 2004 The Neuroanantomy of the Circadian Rhythm.).

In the United States, DSPS is common. Approximately 7-10% of patients who complain of insomnia are diagnosed with a circadian rhythm disorder, most often DSPS. The prevalence of DSPS is probably higher than that because the total sleep time is typically normal in patients with DSPS and because patients with DSPS adjust their lifestyle to accommodate their sleep schedule and do not seek medical treatment. In adolescence, the prevalence is approximately 7%. In contrast, true ASPS probably is quite rare. An age-related phase advance, however, is common in the elderly, who tend to go to sleep early and get up early.

The diagnosis of circadian rhythm disorders is based primarily on a thorough social, physical and neurological history. Differentiation of transient disorders from chronic disorders and primary disorders from secondary disorders influences the direction of evaluation and treatment plans. As with all medical and psychiatric histories, the nature of the complaint is the first order of business. In cases of sleeplessness, distinguishing individuals with difficulty initiating sleep from those with difficulty maintaining sleep, those with significant daytime impairment, and those complaining of nonrestorative sleep is important.

Disorders associated with various sleep disorders include narcolepsy, cataplexy, restless-leg syndrome, and sleep apnea. Anxiety disorders do not include normal emotional reactions, a normal reaction to stress or reactions secondary to an organic cause such as a physical illness or drug exposure.

### E. CNS Disorders

The present invention is also directed to methods of using botulinum toxin based pharmaceuticals injected transcutaneously or by any of the routes of administration disclosed herein, to induce a central nervous system depressive effect for the treatment of various CNS disorders. The inventor has found that botulinum toxin exerts a CNS depressive effect in rats injected transcutaneously in the scalp. The injections are not intracranial or directly into the brain, but may include or specifically exclude intrathecal and intraspinal injection or administration. It is hypothesized that transcutaneous administration of botulinum toxin penetrates the blood/brain barrier. The present invention provides methods for using the botulinum toxin based pharmaceuticals disclosed herein for the treatment of seizures, anxiety, agitation, mania, bipolar disorders, generalized seizures, mental retardation, delirium, hyperactivity syndrome, attention deficit disorder (ADD), dementia, Huntington's disease, Alzheimer's disease, Parkinson's disease, psychosis, schizophrenia, insomnia and other CNS disorders.

In certain embodiments, the botulinum toxin based pharmaceuticals disclosed herein are used at various dosage levels to induce a generalized atrophic effect in the CNS. This effect is useful in the treatment of various CNS disorders. The inventor has found that rats injected with high doses of botulinum toxin (i.e. doses at or near the LD.sub.50) exhibit expanded or enlarged lateral ventricles in their brains. Controls show no such effects while treated animals show a marked effect. Generalized brain atrophy is indicative of biological activity at the level of neurotransmitters that is induced by transcutaneous administration of botulinum toxin. The evidence is consistent with a suppressive effect in the hypothalamus in the treated animals. This could cause direct effects on the release of hormones such as thyroid releasing factors, gonadotropin releasing factor, etc.

All books, articles, patents or other publications and references are hereby incorporated by reference in their entireties. Reference to any compound herein includes the racemate as well as the single enantiomers.

Further Evidence to the Transcranial Movement of Botulinum Toxin Injections from Extracranial Soft Tissue Injections in the Head and Neck Region (See Animal Studies)

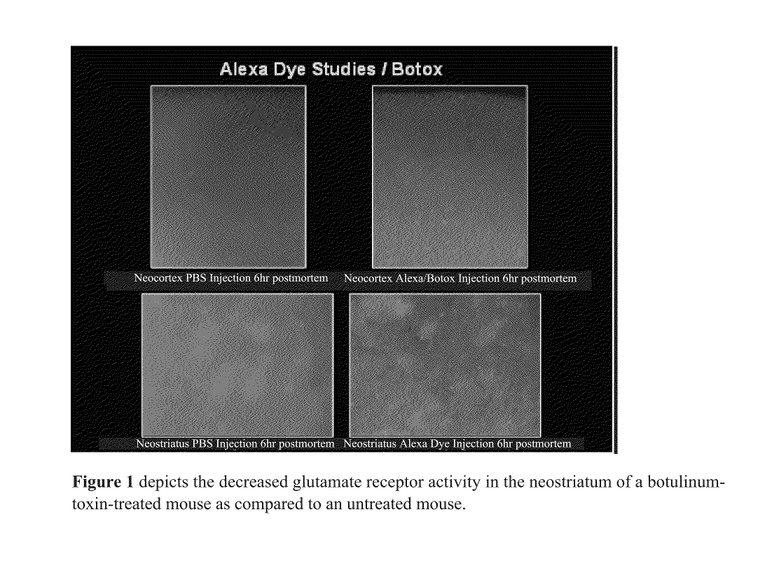

Botulinum toxin has been shown to create a brain atrophy through direct diffusion into the brain based on morphic changes with high-dose extra cranial head injections in animals. Changes in neurotransmission and critical neurotransmitters such as glutamate, norepinephrine, and acetylcholine were markedly altered at doses approaching lethal levels. Scientific foundation of the effect on neurotransmission studies had been conducted with multiple critical neurotransmitters. GABA receptors (see FIG. 1) have been monitored. As glutamate receptors have been decreased on neurons, GABA receptors surprisingly were also noted decreased in intensity and expression over controls after extracranial injection of botulinum toxin in brain structures in the 20 to 30-gram mouse. These receptors are thought to be critical in balancing neurotransmission against glutamate and their expression. The finding that GABA receptors are also blocked has implications in understanding the CNS effect of botulinum toxin. Certain GABA receptors blockers such as benzodiazepines have been noted to be active in the treatment of many diseases, including those of anxiety, general anxiety disorder, anxiety related anorexia, obsessive compulsive personality traits, obsessive and compulsive behaviors, self-mutilating behaviors, as well as posttraumatic stress. Typically anesthesiologists use such drugs to treat pain and agitation prior to a painful medical experience to relieve the pain experience. They have also been used in mood and affect disorders as mentioned herein. The blockage of GABA transmission has an opposite effect that glutamate neurotransmission receptor blockage. It appears that botulinum toxin not only affects glutamate neurotransmission but also has an effect on GABA transmission. The sum total is critical to the modulating and stabilizing effect of extra-cranial injections of botulinum toxin on mood and affect disorders.

### Botulinum Toxin for the Treatment of Central Hypertension

Excess glutamate expression has been associated with excess norepinephrine transmission in the brain and the pathogenesis of central hypertension. Central hypertension is a form of hypertension caused by an abnormality in central nervous system neurotransmitters which results in increased sympathetic outflow from brain and brain stem structures. Glutamate elicited norepinephrine releases have been documented in the literature. There have been studies in the past using antagonists of N-methyl-D-aspartate receptors and glutamate receptors in the modulation of central hypertension. It has been strongly suggested that NMDA receptors and glutamate receptors and other forms of glutamate receptors may have a regulatory effect on central sympathetic tone and hypertension.

The movement of botulinum toxin into the brain to block relative glutamate metabiotrophic receptors and/or NMDA receptors for central hypertension is a concept that is derived from multiple effects of the botulinum toxin on the central nervous system. The mechanism involves,

1. Direct effect on anxiety

2. Direct effect on sympathetic central nervous system outflow

3. Direct effect on promotion sleep and relieving insomnia

4. Direct effect on circadian cycles such as regulation and governance of the angiotensin-renin aldosterone system.

We have seen examples of patients with botulinum toxin injections around the face and eye who had improvements in mood and affect, improvement in depression, and decrease in anxiety also associated with a drop in blood pressure. Alteration in light sensitivity in certain patients indicate a circadian effect which influences the angiotension rennin cycles. Such an observation can be utilized to treat difficult patients with hypertension by steps of:

1. Identifying a patient with central hypertension or a resistant hypertension that use a pharmaceutical technique, that's drugs.

2. Injection of botulinum toxin in the soft tissues outside the neurocranium in a method such as to enhance the diffusion of botulinum toxin into regions of the central nervous system inclusive of regions of the basal ganglia and locus coeruleus, pons, midbrain, and thalamus. Caudate nucleus is also a target of botulinum toxin diffusion and could also be a target for the use of extracranial injections. The targets are within the central nervous system and more specifically in the regions of the brainstem governing autonomic nervous output.

The precise mechanism by which this occurs to block at least one neurotransmitter important in treating centrally mediated hypertension. Such central mediated hypertension can occur as a function of brain disease or it can be idiopathic and part of the components of essential hypertension. Essential hypertension is a target for the treatment of botulinum toxin. That means hypertension not secondary to renal disease, brain disease, or other secondary forms of hypertension.

Up regulation of NMDA receptors activity may also have effects on hypothalamus. Previously taught sleep disturbances with circadian clock abnormalities are also a form of mediation of hypertension. It has been previously thought that botulinum toxin has been useful for treating circadian rhythm disorders and the circadian rhythm disorder of target the hypothalamus which results in improvement in hypertension.

These observations are consistent with a previous notion called The Centurion Effect that represents an effect discovered by the inventor using his 30-year experience of injecting botulinum toxin and noting that patients over the age of 61 who received botulinum toxin repeatedly seemed to live longer than the average person who is followed in the 2007 United States Census.

### The Relationship of Mood, Affect, Anxiety, and Hypertension

As previously taught botulinum toxin is effective for mood and affect disorders consisting of depression (major depression, anxiety disorders inclusive of obsessive compulsive personality traits, posttraumatic stress, as well as general free-floating anxiety and various forms of anxiety neuroses, and sleep disorders including various forms of insomnia). Herein describes a method of treating hypertension utilizing a multi-faceted and multi-approach technique involving:

1. Treating a problem of mood and affect, particularly anxiety, anxiety neuroses, or sleep disorder subsequent with hypertension.

2. Injecting botulinum toxin into the soft tissues outside the neurocranium such that the botulinum toxin diffuses into the central nervous system brain parenchyma affecting at least one neurotransmitter of the brain.

3. Identifying a patient with central hypertension.

4. Assessing the outcome to be an improvement in the systolic and diastolic blood pressure.

Described herein comprises the step also of diagnosing central hypertension. Central hypertension can be diagnosed by identifying an abnormality in at least one neurotransmitter. This neurotransmitter can be norepinephrine, glutamate, glutamate receptors, NMDA receptors, norepinephrine receptors, and norepinephrine neurotransmitter, and acetylcholine receptors and acetylcholine neurotransmitter. More specifically, glutamate neurotransmitter or glutamate receptors inclusive of NMDA receptors.

Given these observations the scanning could involve spectral MRI scans. PET scans, or any other scan form of imaging scans that assays abnormalities of neurotransmitters. The approach would be:

1. Diagnosis hypertension

2. To diagnosis whether hypertension is refractory to conventional therapy, such as the use of beta blockers, ACE inhibitors, ACE receptor inhibitors, calcium channel blockers, or diuretics. If these medicines are not adequate or effective, one could presume that there is a central hypertension in place. Furthermore, and more specifically, it would be possible to diagnosis whether a neurotransmitter abnormality in the level of the brain, brain stem conclusive of diencephalon, mid-brain, pons, or medulla is abnormal. Such an abnormality could include imaging glutamate receptors, glutamate neurotransmission or any other form of neurotransmitter transmission. Once a diagnosis of central hypertension is put forth a dosing between 2.0 and 3,000 LD50 units of type A botulinum toxin could be administered in a single or multi-focal injection strategy so that the soft tissues are injected outside the neurocranium, and more preferably, that the injection sites should correspond to a distance that is closer to critical abnormality areas in the brain so that the botulinum toxin has an ample chance to diffuse into the region using lower doses to mitigate against weakness. These doses can range most preferably to under 800 units of Type A toxin, and more preferably to under 400 units of Type A toxin, and most preferably to under 200 units of Type A toxin. The intent is to create diffusion across the neurocranium, across blood brain barriers into the central nervous system to create an effect, 1. Essential neurotransmitter so that the hypertension can be treated. It is essential to understand that the hypertension is central hypertension and is not caused by other forms of hypertension inclusive of renal vascular hypertension. Essential idiopathic hypertension can be combination of central hypertension and possible abnormalities in kidney function. This is also a target for the proposed invention.

### Targeted Glutamate Receptor Neurotransmitter Related Disease

Glutamate is an important neurotransmitter; it constitutes about 40% to 50% all synaptic transmission occurring in the human brain and spinal cord. It is a critical neurotransmitter that has been noted to be related to cell death and neurotoxicity particularly at high levels. At high levels the glutamate neurotransmission has been associated with an NMDA receptor and glutamate receptor associated flow of ions into the nerve cells resulting in death. There have been extensive efforts to use glutamate receptor blockers for the treatment of multiple central nervous system disease. Ketamine has been associated with the treatment of depression by blocking glutamate receptors and therefore relieving depression. There have been a number of efforts to use glutamate receptor blocking for a number of central nervous system disease processes. The use of glutamate receptor blockers after stroke as a neuroprotector has been tried. Because multiple with side effects of a number of test drugs acting as glutamate receptors blockers, this approach to date has failed. A safe and effective glutamate receptor blocker will be needed to treat a number of central nervous system diseases that have a low toxicity. The unique property of botulinum toxin's ability to move through the blood brain barrier into the central nervous system and effectively down regulate both glutamate receptors and also block glutamate neurotransmission by other mechanisms would clearly be an advance in the therapy for a number of central nervous system diseases. The established high safety profile of botulinum toxin reinforces the applicability in many clinical situations calling for neuroprotection described herein.

These central nervous system diseases involve diseases or disorders of mood and affect including major depression, bipolar disease, bipolar depression, bipolar mania, mania, schizophrenia, psychosis, dementia, Alzheimer disease, Huntington's disease, Parkinson's disease, obsessive compulsive personality traits, recurrent epilepsy, forms of anorexia, autoimmune central nervous system diseases involving glutamate such as systemic lupus erythematosus with central nervous system involvement, head injury, head trauma, brain contusion, ruptured cerebral aneurysm, hydrocephalous, or any other central nervous system disease that involves destruction of tissue.

It is important that glutamate can be imaged with spectral MRI, PET scans, or any other methods. Various methods and techniques demonstrating glutamate or any other neurotransmitter to be at a higher level in the disease process presents as an excellent opportunity for the administration of botulinum toxin to be administered to protect against neurotransmitter associated neuronal toxicity.

Herein describes a method of treating and preventing the progression of central nervous system diseases involving administration of botulinum toxin in the extracranial portion of the head. This can include scalp, face, periocular region, and head and neck, mandibular region, periotic regions or nasal sinuses, nasal cavities, sinuses, bone structures and oropharynx to achieve an adequate diffusion of botulinum toxin into the brain. Inherent in this invention is the direct diffusion of botulinum toxin into the brain and causing a blockage of one important neurotransmitter. Previously taught by others is that botulinum toxin is blocked from penetrating the blood brain barrier. Herein describes an opposite and novel observation that botulinum toxin does get into the brain and can have a direct effect on neurotransmitters. More specifically, neurotransmitters include glutamate, acetylcholine, glycine, GABA, and it can affect expression of the appropriate receptors to neutralize toxic neurotransmitters from causing cell death.

The method involves:

1. Identifying a central nervous system condition.

2. To apply botulinum toxin into the extracranial soft tissues in order to diffuse into the brain.

3. To block at least one neurotransmitter in the brain and therefore improve symptoms or decrease the progression of the central nervous system disease.

4. To assess the results of the injections with neuro imaging and clinical history and physical exam.

5. To have repeated injections given and monitoring for the effect so that progression of the central nervous system disease does not occur.

Again, the central nervous system disease can be neurodegenerative in nature, cerebrovascular in nature, traumatic in nature. Protection can occur at the level of cranial nerves. Neuroprotection can function in relations to infection with viruses or bacteria, or a hemorrhage of the brain.

In summary, the invention can be described as:

1. Identifying a central nervous system disease with assessment of an imaged neurotransmitter abnormality.

2. Applying botulinum toxin in the extracranial soft tissues of the head and neck.

3. Assessing an improvement in the symptoms produced by the central nervous system disease or decreasing the rate of progression of the central nervous system disease.

### Dosing

Dosing again is between 2.5 and 3000 units. The dosing would involved would be placed in multi-focal locations. Selective administration into the brain is possible by injecting over areas of the brain targeted. For instance, if the brain stem and mid-brain is targeted periocular injections would be preferred. If the sinus injections could be given, as well as nasal cavities sinus injections can be given for this region. If the target is into the temporal lobe they could be given over the temporal bone periotic areas. If it is in the occipital lobe they can be given under the neck over the foramen magnum over the occipital bone so that diffusion can be accomplished through the diploic veins in that part of the head. It should be noted that venous drainage into the brain is often in the soft tissues within the extra cranial circulation that is injected, but the mechanism may not be limited to this route of entry. Direct diffusion of a small amount of toxin through diffusion and through soft tissues, and even bone, is possible. A guidance map demonstrating the venous drainage of the extra neuro cranial regions of the head and neck is given in FIG. 1.

The injections need to be repeated at 3 to 4-month intervals as necessary related to the progression of the disease for the improved symptomatology. Coincident imaging may be done for a neurotransmitter. Specifically, the neurotransmitter can be a select version of glutamate, acetylcholine, GABA, glycine, or norepinephrine.

Relative proportions of neurotransmitters may be assessed with different imaging techniques so that the relative proportionality of the neurotransmitters may be assessed, and diagnostically matched to both botulinum toxin effect and pathogenesis of the targeted disease.

This application of the invention should be considered to be specifically matched with an enabling diagnosis and an associated neurotransmitter abnormality. Mood and affect disease is a general term inclusive of major depression; bipolar disease; minor depression; reactive depressions; schizophrenia; seasonal affective disorder, generalized anxiety disorder, anxiety neurosis with subcategories, post-traumatic stress syndrome. Further anxiety related anorexia, anorexia nervosa, psychoses; obsessive personality traits; recurrent epilepsy of all forms including generalized seizures, partial complex seizures, focal motor seizures; and any toxic metabolic disorders occurring in the central nervous system can be targets of a regional neurotransmitter abnormality.

### Preferential Injection Strategy

Because it is now known that there is botulinum penetration and diffusion influencing neurotransmitters of the brain, it is possible to alter the injection strategy so that specific portions of the brain to receive a preferential dosing of the botulinum toxin. Preferential dosing in different regions of the brain would involve using a graduated quantity of botulinum toxin in different regions within soft tissues outside of the neurocranium so that the diffusion gradient is optimized to the region of the brain which the central neurotransmission is to be targeted. This would mean for example, the periocular region would more likely in supraorbital region would almost likely diffuse into the areas of the mid-brain, frontal lobe, and thalamic region based on anatomic drainage patterns of the venous structures in the area to the brain, as well as a close contiguous location. Treatment of temporal lobe areas would be involved in injections over the temporal bone. Injections of the cerebellum would involve injections over the posterior occipital bone which overlies the posterior fossa and cerebellum. Injections into the upper cervical spinal cord would involve injections over the paraspinal bone processes, the spinal processes overlying the spinal cord.

This selective injection is both anatomically driven based on delivery and can also be optimized by dosing. As previously stated, the importance of the extracranial injection makes this an amenable to office-based treatment with limited side effects and limited damage to critical structures of the central nervous system, such as cortex and brain structures. This invention does not teach direct injections into the brain by any such techniques. In order to enhance injections it is possible for adjuvant therapy to include using small screw conduits in critical areas of the neurocranium that could allow for a greater diffusion if needed to create more of an effect in a given region.

The unique observations of what is taught herein are that central neurotransmission can be controlled with injections of botulinum toxin outside the neurocranium by injections at the soft tissue. As the inventor has previously taught on other patents the diffusion of botulinum toxin is a dose-dependent phenomenon and the accessibility of diseased brain tissue by injections into soft structures outside the neurocranium represents a novel access to the central nervous system that was not previously appreciated and the technology previously practiced in any way. With the advance of advanced imaging of neurotransmitters this form of invention and injection strategy is a novel way of dosing administration and targeting diseases as taught previously. These diseases can include neurodegenerative diseases which include Alzheimer's, Parkinson's disease, Huntington's disease, seizure disorders, recurrent seizure disorders with damage, and mood and affect disorders, and damage from a cerebral vascular occlusion. Limiting the damage for cerebral vascular occlusion involves limiting the excretion of one critical neurotransmitter. This neurotransmitter more specifically can involve glutamate or glutamate related activities at the central synaptic level. So what is taught here in practicing this invention is:

1. To identify the area of the brain which there is a focal area of neurotransmitter disease and activity by imaging studies.

2. Applying the botulinum toxin in a closer proximity to this region by direct injection or by other forms of transdermal delivery system as taught in other parts of this disclosure.

3. To assay the clinical benefits from such an injection strategy.

4. To repeat the scanning structures or scans to see if the neurotransmitter hyperactivity has been duly suppressed. Repeated injections over many years is anticipated.

### Targeted Brain Regions Using Botulinum Toxin and Unique Injection Strategies.

As botulinum toxin can diffuse directly into the neurocranium, through venous drainage systems or other conduits, and penetrate the blood brain barrier, it is possible to target regions of the brain with abnormally high levels of glutamate neurotransmission. This technique to date has not been previously described and offers a unique method of dosing and altering regional brain neuro transmission which can be applied for various central nervous system disorders described herein. In recent technological advancing in brain imaging, various neurotransmitter imaging can be accomplished with studies using PET scans (Positron Emission Tomography) and Spectral MRI (Magnetic Resonance Imaging). Other methods are anticipated. Such advances are useful in beginning to assess the contribution that defects in various transmitters play in characterizing and understanding mechanisms in many disorders of mood and affect (major depression, bipolar disease, mania, schizophrenia, Parkinsons disease, Alzheimer's disease, anxiety, post traumatic, stroke, brain hemorrhaged, circadian rhythm disorders (sleep disturbances, hypertension, insomnia, hypersomnia, sleep apnea), anxiety disorders, post traumatic stress syndromes, chronic anxiety, anorexic disorders, excessive appetite). These imaging methods not only characterize abnormalities in neurotransmission, the methods can allow a geographic, localization of central nervous system areas where the neurotransmission is abnormal. Such localization methods leads to a structured blueprint as to where extracranial injections can be administered to allow for maximizing penetration of the botulinum toxin based formulation into the neurocranium, through the blood brain barrier, so that targeted regions receive administration of the drug, minimizing diffusion away from areas of muscle, peripheral and central nerves in which drug application would be undesirable and cause side effects, such as impaired facial expression, ptosis, inability to close eyelids, head movement weakness, drooling, brow ptosis, paralytic ectropion, facial asymmetry, facial palsy, diplopia, slurred speech, swallowing difficulty, upper airway incompetence, aspiration, sad face appearance, or other side effect related to excessive dosing of muscular tissue in the head and neck region.

The characterization of neurotransmitter abnormalities can be applied to the aforementioned diagnostic entities to further characterize the nature of the disorder.

The characterization shall be not only by neurotransmitter type but also the specific region of the brain where the abnormal neurotransmitter activity is occurring. For instance, if abnormal activity in a neurotransmitter X is found in region Y of the brain, injection of botulinum toxin into soft tissues outside the neurocranium can be fashioned to target preferential penetration and diffusion into region Y to block neurotransmitter X.

Region Y can be any one or combination of:

Diencephalon (rostral brainstem, caudate nucleus, thalamus, lenticulate nucleus), midbrain, pons, medulla, any specific gyri in either right or left brain hemispheres, hypothalamus, pituitary glands, frontal-parietal-temporal lobes, spinal cord, and occipital lobes or any other portion of the central nervous system.

Neurotransmitter X can be any one or combination of glutamate, acetylcholine, gamma amino benzoic acid (GABA), nor-epinephrine, glycine, serotonin or dopamine. More specifically, glutamate, acetyl choline GABA, or nor epinephrine.

Injection may be anywhere outside the neurocranium which maximizes diffusion and central nervous system delivery either by transcranial delivery by penetration through venous drainage (orbital veins, facial veins, diploic veins), or any other constituents of the blood brain barrier. Distance analysis from injections sites can be easily cataloged using MR imaging or CT imaging with computer measurements over imaged anatomy.

The central nervous system diseases involve disease or disorders of mood and affect including major depression, bipolar disease, bipolar depression, bipolar mania, mania, schizophrenia, psychosis, dementia, Alzheimer disease, Huntington's disease, Parkinson's disease, obsessive compulsive personality traits, recurrent epilepsy, autoimmune central nervous system diseases involving glutamate such as systemic lupus erythematosus with central nervous system involvement, head injury, head trauma, brain contusion, ruptured cerebral aneurysm, hydrocephalous, or any other central nervous system disease that involves destruction of nervous tissues.

Intrinsic in this invention is a notion that glutamate can be imaged with spectral MRI, PET scans, or any other mechanism that shows glutamate or any other neurotransmitter to be at a higher level, and that such neurotransmitter has been associated with toxic effect on the neuron.

Herein also describes a method of treating, slowing or preventing the progression of central nervous system diseases involving administration of botulinum toxin in the extracranial portion of the head to prevent, retard, slow or even arrest the progression of any central nervous system disease by blocking neurotoxin neurotransmitters using extracranial botulinum toxin.

Injection areas can include scalp, face, periocular region, and head and neck, mandibular region, periotic regions or nasal sinuses, nasal cavities, sinuses, bone and oropharynx to achieve adequate diffusion of botulinum toxin into select regions in the brain. The process of exacting the location is dependent on analysis of the areas of the brain demonstrated to have abnormalities in neurotransmission by aforementioned techniques and selecting the extracranial region of the head and neck which will allow maximal delivery. Inherent in this invention is the direct diffusion of botulinum toxin into the brain via transcranial passage (without direct brain injection) and causing a blockage of one important neurotransmitter. Previously taught by all others is that botulinum toxin is blocked from penetrating the blood brain barrier. Herein describes an opposite view to prevailing thought and novel observation that botulinum toxin does get into the brain by extra cranial injection within hours to days after injection and can have a direct effect on neurotransmitters. More specifically, neurotransmitters include glutamate, acetylcholine, glycine, GABA, and it can affect expression of the appropriate receptors to neutralize toxic neurotransmitters from causing cell death.

The method involves:
1. Identifying a central nervous system condition by diagnosis (diagnostic entities). Identifying the region of the brain which the neurotransmitter is determined to be abnormal for the select diagnosis
2. Applying botulinum toxin into the extracranial soft tissues in quantities sufficient to diffuse into a targeted region of the brain or in certain circumstances diffusely throughout the brain.
3. Blocking at least one neurotransmitter in the brain and therefore improve symptoms or decrease the progression of disease.
4. Assessing the results of the injections with neuroimaging and clinical assessment of signs and symptoms. Neuro-imaging may have been previously used to establish the location of injection or the nature or subtype of the diagnostic entity being treated.
5. Repeating injections given and monitoring for the effect so that progression of the central nervous system disease does not occur.

### Representative Table for Access to Brain Neurotransmission:

The following represents an access table for injection of botulinum toxin to access various areas of the brain with attendant diagnostic condition for which the targeted administration is intended. This table is intended for approximation and example. The neuroimaging of each individual with a disorder of neurotransmission would need to be assessed prior to application.

| Region of injection in | | |
|---|---|---|
| close proximity to region of | Location of Abnormality of | |
| the brain targeted | Neurotransmission | Example of Condition |
| Face, Sinus, nasal mucosa, | Upper brainstem and medial | Circadian rhythm disorder |
| oropharynx, nasopharynx | hemispheres | Mood and affect disorders, |
| | | Light sensitivity disorders, |
| | | various chronic pain |
| | | syndromes |
| | | Stroke in brainstem, |
| | | Parkinson's disease |
| Scalp | Hemispheres and cerebral | Epilepsy, seizures, dementia, |
| | cortex | Alzheimer's, Stroke, cerebral |
| | | hemorrhage |
| Specific region of the scalp | Areas of abnormalities | Cortical and cerebral |
| | within selected gyri on each | vascular insufficiency, |
| | cerebral hemisphere | cortical stroke, cortically |
| | | generated seizure disorder, |
| | | Cognitive disorder, memory |
| | | disorder |
| Occipital region Back of | Cerebellum, occipital lobe | Stroke, |
| Head | | |
| Cervical Bony spine in back | Cervical spinal cord | Nerve root compression, |
| of neck region | | referred neck pain |

More specific injections can be give in regions where individual gyri, or very specific focal areas of the brain can be treated by extra cranial injections. For example if focal motors seizures are emanating from the prefrontal cortex from the left cerebral hemisphere, injection over the neurocranium in this region can be accomplished for direct administration without causing effect to contiguous regions.

The above table is only an example and can be extended based on conventional anatomy as described by Grant's atlas of Anatomy and Gray's Anatomy and Functional neuroanatomy textbooks. In clinical practice, localization is best done by use of neuro imaging studies, in particular spectral MRI and PET scanning or any other form of localizations technique that identifies the condition as regional in the brain. In generalized brain or spinal cord pathologic conditions, diffuse injections strategies can be used and customized to produce a more general effect on the brain.

### Further Perfections of Injection Strategy

Because it is established that that there is a botulinum penetration into the brain and a resultant effect on neurotransmitters, it is possible to alter the injection strategy so that specific portions of the brain receive a preferential dosing of botulinum toxin. Preferential dosing in different regions of the brain would involve using a graduated quantity of botulinum toxin in different regions within soft tissues outside of the neurocranium so that the diffusion gradient is optimized to the region of the brain which the central neurotransmission is to be targeted. This would mean for example, injecting the peri ocular region would more likely diffuse into the areas of the mid-brain, frontal lobe, and thalamic region based on anatomic drainage patterns of the venous structures in the area to the brain, as well as a close contiguous location. Treatment of temporal lobe areas would be involved in injections into soft tissues over the temporal bone. Injections of the cerebellum would involve injections over the posterior occipital bone which overlies the posterior fossa and cerebellum. Injections into the upper cervical spinal cord would involve injections over the paraspinal bone processes, the spinal processes overlying the spinal cord.

This selective injection is both anatomically driven based on delivery and can also be optimized by dosing. As previously stated, the importance of the extracranial injection makes this an amenable to office-based treatment with limited side effects and limited damage to critical structures of the central nervous system, such as cortex and brain structures. This invention does not teach direct injections into the brain by any such techniques.

In order to enhance injections, it is possible for adjuvant therapy to include using small screw conduits in critical areas of the neurocranium that could allow for a greater diffusion if needed to create more of an effect in a given region.

### Implantable Conduit Pins to Enhance Transcranial Diffusion:

As transcranial diffusion is necessary for delivery of botulinum toxin into the brain structures through bone and blood brain barrier, there are methods available to enhance this process which can be minimally invasive and not requiring any major neurosurgical procedure. These methods would involve implantation of a device which enhances direct penetration of botulinum toxin through the neuro cranial bony vault, through the three layers of meninges (dura, arachnoid, and pia) and into the brain substance in targeted locations or diffuse application. For example, a titanium pin with open canals can be fashioned into a self-drilling screw and placed into the out and middle and in some circumstances the inner tablet of the skull. Such implants enhance the natural canals seen in the bony skull around venous and allow more toxin penetration if injected over these implants. These devices may be made out of any biocompatible materials (titanium, ceramic, plastic, plastic) which are bio compatible. They can be left in position even when no future injections are required.

The use of conduits to help central nervous system permeation allow lower doses to be used to achieve higher brain levels of neurotoxin without causing excessive weakness outside the central nervous system from diffusion of its neuromuscular effect.

### Focal Effects in Dementia and Other Forms of Neurodegeneration:

The unique observations of what is taught herein are that central neurotransmission can be controlled with injections of botulinum toxin outside the neurocranium by injections at the soft tissue. As the has previously taught in referenced patents, the diffusion of botulinum toxin is a dose-dependent phenomenon and the accessibility of diseased brain tissue by injections into soft structures outside the neurocranium represents a novel access to the central nervous system that was not previously appreciated or previously practiced in a directed fashion. With the advances in imaging neurotransmitters this form of invention and injection strategy is a novel way of dosing administration and targeting diseases as taught previously. These diseases can include neurodegenerative diseases which include Alzheimer's, Parkinson's disease, Huntington's disease, seizure disorders, recurrent seizure disorders with damage, and mood and affect disorders, and damage from a cerebral vascular occlusion. Limiting the damage for cerebral vascular occlusion involves limiting the critical neurotransmitters involved in neuronal cell damage. This neurotransmitter more specifically can involve glutamate or glutamate related activities at the central synaptic level. So what is taught here in practicing this invention is:

1. To identify the area of the brain which there is a focal area of neurotransmitter disease and activity.

2. Applying the botulinum toxin in a closer proximity to this region by direct injection or by other forms of transdermal delivery system as taught in other parts of this disclosure.

3. To assay the clinical benefits from such an injection strategy.

4. To repeat the scanning structures or scans to see if the neurotransmitter hyperactivity has been duly suppressed.

### Insomnia

It has been noted that insomnia is associated in the past with certain problems of circadian rhythm function. Both circadian rhythm functions of sleep, blood pressure regulation, cortisol secretion in systemic circulation are to a degree interrelated with the central nervous system circadian system.

From a medical point of view disorders of insomnia have been associated with a difficulty with blood pressure control. Sleep disturbances can be associated with high blood pressure in certain populations of patients with resistant hypertension. In these populations it is possible to use botulinum toxin in a form of extracranial soft tissue injection to affect the central nervous system in such a way that the neurotoxin can mitigate sleep disorder and therefore mitigate hypertension. A mechanism by which botulinum toxin can work for hypertension is:

1. Improve sleep disorder.

2. Improve anxiety.

3. Improve major depression.

4. Have an effect on glutamate central nervous system neurotransmission so that the circadian clock and circadian rhythms are better maintained.

The above complex of effects requires that the botulinum toxin move from extracranial injection location through the neurocranium and affect at least one neurotransmitter. The neurotransmitter most likely would be glutamate but can be norepinephrine, acetylcholine, or other forms of neurotransmitters. The diffusion into the brain is critical in how this invention works. It specifically does not work by virtue of affecting a sensory nerve, having effect on sensory nerve adaptations, or limiting any form of neuromuscular affect to cause weakness. In practicing the invention the following should be considered:

1. Diagnosis of hypertension resistant to conventional use or primary hypertension without prior treatment.

2. Diagnosis of a concomitant problem with sleep.

3. Administration of botulinum toxin for the treatment of the sleep disorder thereby reconciling the hypertension.

4. Monitoring the hypertension using standard monitors and procedures.

The above appreciates the central nervous system contribution to idiopathic hypertension, as well as other forms of hypertension.

Sleep disorders have been associated with high incidents among minorities. The sleep disorders are more likely to occur in obesity, and also have a racial configuration to be more prone in the black population. Such populations are also at more risk for cardiovascular disease, so race and ethnicity are important in helping predict the utilization of the invention described herein. In this situation an assessment would need to be made in high-risk populations such as a racial preference with African Americans having much higher incidences of both sleep disorder and cardiovascular disorders. Such differences can be exploited to focus the practice of the invention on populations of high risk. Furthermore, as this invention is a physician-administrated technique and does not require any patient compliance, the invention can be used in patients where use of medical compliance is limited.

In these situations the physician administers the toxin at a dose between 2.5 units and 3000 units of Type A on a regular basis. The patient is not required to take a daily medication at a specific time, so the compliance factor is limited. The unique duration of action of botulinum toxin being very long on the matter of 12 to 16 weeks can be helpful.

### Administration in Depression, Unique Attributes

### (Major Depression, Bipolar Depression, Seasonal Affective Disorder, Post-Traumatic Stress Syndrome)

Herein it teaches the use of botulinum toxin for depressive disorders, particularly major depression disorder (MDD). It also can be used for bipolar depression, posttraumatic stress syndrome, and various other forms of depression such as seasonal affective disorder.

Herein describes a method that the botulinum toxin is administered as an advantage in that it requires no compliance of the patient on a daily basis. There is also in depression it limits the risk of suicide because the administration is totally physician based and does not require the patient to have access to a large amount of oral medicines.

In essence the suicide risk of botulinum toxin administration is essentially null. This has a huge advantage in treating depression patients. Herein describes a method of not only of treating major depression but treating major depression with a high suicide risk. In the practice of the invention the psychiatrist or the physician that assesses the suicide risk of the patient based on his past history, prior attempts at suicide, and prior compliance with the use of the medication. If a risk is determined then botulinum toxin is recommended. The inventive nature of this relies on the fact that botulinum toxin is unilaterally given by the physician and requires no patient cooperation, as far as dosing or use. In this situation this represents a significant advantage in utility and is inventive in that it solves a major problem within the practice of psychiatry.

### Hypertension, Glutamate and Circadian Disorders:

Blood pressure regulation is a major circadian function with diurnal variation.

Hypertension can be created by abnormalities in central nervous system functions and its treatment can be modulated by use of CNS acting agents, such as botulinum toxin preparations. Excessive sympathetic outflow from autonomic CNS centers are governed by circadian mechanisms which can have pathologic consequences. Myocardial infarctions are most commonly experience in mornings in part because of diurnal increases in morning blood pressure and cardiovascular stimulation. Mitigating sympathetic output via trans cranial diffusion of botulinum toxin into circadian clocking mechanism in the hypothalamus can in effect be helpful to modulate blood pressure control.

The periventricular nucleus (PVN) of the hypothalamus contributes to the generation of hypertension in rat experimental models. A population of neurons project into the sympathetic neurons in the spinal cord and rostral ventrolateral medulla and regulate sympathetic outflow and vascular tone and blood pressure. Elevated sympathetic activity is a contributing cause of essential hypertension, such as spontaneous hypertensive rat models. Glutamate is the primary neurotransmitter in the paraventricular nucleus. Glutamate is the major neurotransmitter in the paraventricular nucleus and increased glutamate input into the pre sympathetic neurons has been shown to cause increased vasomotor tone and hypertension in rat models. The paraventricular nucleus is part of the circadian neural structures with significant input from the suprachiasmic nucleus.

Injections directed at extra cranial structures which maximize flow into the hypothalamus (0074), basal ganglia, pituitary vascular system in order to modulate glutamate neurotransmission in these regions is effective in regulating and suppressing sympathetic output from these regions, such as within the paraventricular nucleus to regulate blood pressure. Glutamate in these region has been associated with circadian rhythms (Castanada T R Circadian Rhythms of dopamine, glutamate, and GABA in the striatum and nuceus accumbens in the awake rat, modulation by light J Pineal Res 2004 36(3), 177-85.

Therefore, the use of botulinum toxin to the face forehead and extracranial region that maximize venous drainage and transport of botulinum toxin to brain areas governing circadian rhythm are targeted using single or multiple injections to treat central hypertension and more specifically hypertension governed by circadian diurbnal variation. This form of hypertension disorder has been shown to be more dangerous relative to cardiovascular risk of myocardial infarction. Dips in blood pressure during sleep are often obliterated in this form of hypertension. Botulinum toxin, by treating the circadian rhythm disorder effectively restores the diurnal blood pressure variation and mitigates hypertension. Doses may be as described herein.

### Circadian Rhythm Disorders:

Circadian rhythms are subject to variations and expressions of various neurotransmitters and associated receptors. Various circadian rhythm disorders can result from pathologic variations of neurotransmitters based on sensory light deprivation (blindness), acquired disorders of neurotransmitters, and genetic abnormalities in enzymes, mechanical proteins, receptors and other components of the molecular basis of neurotransmission.

| Function | Abnormality | Syndromes |
|---|---|---|
| Sleep | Insomnia, hypersomnia, | See specifications |
| | delayed sleep, | |
| Appetite | Anorexia, obesity, | Genetic related obesity, |
| | Hyperphagia, cortisol | diabetes, Insulin |
| | release | resistance, anorexia |
| | | nervosa |
| Blood pressure | Centrally Mediated | Essential hypertension, |
| | Hypertension, Sodium | Congestive heart failure, |
| | retention | angina pectoris |
| Glucose | Hyperglycemia | Diabetes |
| Metabolism | | |
| Alertness | Chronic fatigue, depression, | See specifications |
| | anxiety, jet lag, attention | |
| | deficits (ADD). social | |
| | function | |
| Thermoregulation | Night sweats, | Menopause related |
| | | symptoms |

Altering function of the circadian rhythm can effectively treat a number of associated disorders such a sleep disturbances, attention deficit disorders, hypertension, cardiovascular stress by sympathetic central nervous system outflow, appetite regulating weight and body fat quantity, and emotional degenerated physiologic dysfunctions. Emotional degenerated dysfunctions include diarrhea associated with anxiety and stress (spastic colon disease), hyperventilation, diffuse sweating, heart rate-palpitations, asthma, increased pain, dizziness, chest discomfort, inattentiveness, forgetfulness, and fatigue.

Improving sleep, sleep synchronization effectively help with many conjoined circadian functions and therefore has been helpful in treating associated dysfunctions. For instance, improving sleep synchronization and associated blood pressure regulation can be helpful in treating hypertension, symptoms of coronary artery occlusion, asthma, functional abdominal pain, spastic colon, ADD, and generalized non disrupt fatigue syndromes.

Improvement in sleep synchronization can improve major depression, mania, bipolar disease, anxiety disorders, migraine related disorders, chronic tension headache, back pain, or any other chronic pain disorder. Improvement in sleep can effectively improve

1. pain syndromes

2. Other disorders of mood and affect including major depression, anxiety syndromes as defined in the DSM IV

3. Generalized fatigue

4. Organic brain symptoms, dementia, memory and cognition

5. Impaired energy and interest in daily life functions

6. Impaired concentration

7. Pathologic Alterations in appetite

8. Excitability and pathologic agitation

9. Recurring thoughts

10 Emotional components to essential hypertension

### Use of Botulinum Toxin for Eating Disorders

As previously described, herein botulinum toxin can be used for the treatment of certain forms of eating disorders. These eating disorders include eating disorders associated with generalized anxiety, phobias, posttraumatic stress syndrome, and forms of anorexia associated with depression. Anorexia nervosa is also a contemplated indication for botulinum toxin if associated with appropriate forms of psychosocial and mood and affect disorders can also be targeted for treatment.

Circadian rhythms not only involve the maintenance of blood pressure, sleep, and other forms of circadian rhythm functions, but they also are implicit in governing the intake of food. The glutamate neurotransmitters, as well as other forms of neurotransmitters can be important to the generation of appetite and anorexia. Herein describes a method of treating patients with certain forms of anorexia that can be associated with abnormalities and neurotransmission of the central nervous system. The application is again through an extracranial injection strategy at doses that are conventionally used such as 5 to 2000 units, more specifically 100 to 300 units, more specifically about 150 to 200 units.

These injections are given in single and multi-focal locations and subcutaneous, subdermal, transcutaneous, intraosseous injections. The injections can be given in a way to focally administer the toxin so that the diffusion is targeted over important areas governing appetite and the brain is going to include the hypothalamus and thalamus, areas of the rostral, and brain stem and mid-brain stem. This can be used in conjunction with other forms of anorexic therapy and can be useful to control anorexia and nausea and appetite.

### Down Regulation of Opposing Receptors:

Described herein is an effect on opposing neurotransmitters glutamate and GABA. The duo activity by down regulating the receptor expression on central based neurons is a very unique attribute in how botulinum toxin acts within the central nervous system. As glutamate is associated with neural hyperactivity and GABA as a neuronal depressor, botulinum toxin modulates both functions of excitability and repression of neuronal biologic activity. Such an effect can have unique properties relative to mood and affect, inclusive but not limited to experiencing extremes of anxiety agitation in one sense and depression at the other extreme.

### EXAMPLES

The following Examples serve to further illustrate the present invention and are not to be construed as limiting its scope in any way.

### Example 1

A 78-year-old male who noted sleep disturbances and anxiety was initially diagnosed with blepharospasm. Botulinum toxin was administered by injection, and the subject noted improved sleep and reduced anxiety.

### Example 2

A 44-year-old bus driver was diagnosed with hemifacial spasm and reported symptoms of anxiety. Botulinum toxin was administered by injection. The subject noted a better ability to cope with work-related stresses and cope with difficult situations with less stress.

### Example 3

A 72-year-old consultant diagnosed with hemifacial spasm who reported sleep disturbances and anxiety was treated with botulinum toxin that was administered by injection. The subject reported improved sleep and reduced anxiety and less agitation.

### Example 4

A 45-year-old woman was treated for cosmetic indications with botulinum toxin. The initial diagnosis was cosmetic rhytides. The subject noted fewer symptoms of depression and less anxiety for a period of two months.

### Example 5

A 44-year-old woman diagnosed with severe tension headaches and sleep disturbances was treated with botulinum toxin by injection. The subject noted improved sleep patterns and fewer headaches up to two months after treatment.

### Example 6

A 73-year-old male with essential blepharospasm reported sleep disturbances and anxiety characterized as “nervous tension.” Botulinum toxin was administered by injection. The subject noted less anxiety and improved sleep after the injections. The reduced symptoms lasted two to three months and ultimately recurred.

### Example 7

A 43-year-old person with myofacial pain and sleep problems was treated with botulinum toxin by injection. The subject noted better sleep patterns after injections that lasted three months.

### Example 8

A 42-year-old person was diagnosed with myofacial pain, tension headaches and depression and treated with botulinum toxin administered by injection. The subject noted some improvement in sleep pattern after the toxin injections.

### Example 9

The subject is a 54-year-old person diagnosed with essential blepharospasm and depression. Botulinum toxin was introduced by injection. The subject noted fewer symptoms of depression after the botulinum toxin injections.

### Example 10

The subject is a 57-year-old physician diagnosed with essential blepharospasm. Botulinum toxin was introduced by injection. The subject noted a feeling of euphoria, well being and improved mood after the botulinum toxin injections.

### Example 11

A 47 year old woman with a history of cervicogenic headache and frequent problems of insomnia. The insomnia was characterized by difficulty initiating sleep, intermittent awakening, early-morning awakening, and inability to maintain sleep. Injections were given in the regions generally used to treat spasmodic torticollis as well as in multiple locations along the hairline, both anterior and posterior. Doses ranged between 5-20 units per subcutaneous injection site with a total dose of 100 U. Within 3-5 days, improvement in the insomnia occurred and lasted between 10-14 weeks. Improvement in each component of her sleep disorder occurred.

Recurrence of the sleep disorder occurred after the 10-14 week period.

### Example 12

A 52 year old woman received botulinum injections for the effacement of glabellar rhytides (facial wrinkles). Further injections were given in multiple locations along the hairlines, she also suffered from insomnia with difficulty initiating sleep and sustaining sleep. After injection with botulinum toxin, sleep pattern improved and lasted the duration of about 10-12 weeks. Total dose administered in multiple locations was 30 Units.

### Example 13

A 71 year old man with essential blepharospasm was injected with 60 U divided along the peri-ocular region and the forehead. Improvement in sleep pattern characterized by more continuous sleep was noted after each injection. The benefit lasted about 3 months and has been noted over 3 injection cycles. When brought to the patient's attention, he associated the improvement to the botulinum toxin injections. Insomnia recurred when he felt the time for repeat injection with botulinum toxin.

### Example 14

A botulinum toxin composition is prepared from any immunotype (A-G) consisting of monocomponent neurotoxin molecules free of accessory or complex proteins, containing human serum albumin, and a nanoemulsion, with various charges. The nanoemulsion may contain polymers consisting of any of the following: polyethylene glycol, vegetable oil, a vegetable oil derivative or a monounsaturated or polyunsaturated oil. The pH may be altered in the preparation to enhance permeability. Alternatively, botulinum toxin is prepared from immunotypes A-G consisting of a monocomponent neurotoxin, without a nanoemulsion carrier, albumin and an acidic pH between 1-6 units. The effect on the central nervous system from transcutaneous injection was demonstrated using a rodent animal model typically used for research in neurodegenerative disease (20-30 gram mice). Injections were given over the scalp region with botulinum type A toxin at a dose close and approximating the LD.sub.50 for this animal. Surviving animals were subjected to autopsy and serial brain cutting and histologically stained using a standard Nissle formula. Substantial atrophy of basal ganglion and periventricular cells was noted. Such changes are not usually seen with systemic illness without direct brain pathology. The neuropathologic assessment is that direct suppressant effects do occur within the central nervous system at high dose (close to the LD.sub.50 for the animal model). More subtle changes are anticipated and seen at lower therapeutic doses based on clinical observations of efficacy for insomnia, dysmenorrhea, depression and anxiety. The experimentation described herein indicates blockage of neurotransmission usually of excitatory neurotransmitters to the extent that pathologic change occurs in brain structures. The major central nervous system neurotransmitters blocked include glutamate, norepinephrine, acetylcholine. GABA effects are augmented. SNAP-25 is noted to be cleaved throughout the targeted areas.

### Example 15

The effect on the central nervous system from transcutaneous injection was demonstrated using a rodent animal model typically used for research in neurodegenerative disease (20-30 gram mice). Four injections of botulinum toxin (totaling 0.8 LD.sub.50 units) were given over the scalp region. Surviving animals were subjected to autopsy and serial brain cutting and histologically stained using a standard Nissle formula. Substantial atrophy of basal ganglion and periventricular cells was noted. Substantial decrease of cholinergic neurons was noted. Substantial decrease in the amount of choline acetyltransferase was noted. More subtle changes are anticipated at lower therapeutic doses based on clinical observations of efficacy for insomnia, dysmenorrhea, depression and anxiety. The experimentation described herein demonstrates blockage of neurotransmission usually of excitatory neurotransmitters to the extent that pathologic change occurs in brain structures. The major central nervous system neurotransmitters blocked include glutamate, norepinephrine, and acetylcholine. GABA effects are augmented. SNAP-25 is noted to be cleaved throughout the targeted areas.

### Example 16

The effect on the central nervous system from transcutaneous injection was demonstrated using a rodent animal model typically used for research in neurodegenerative disease (20-30 gram mice). Four injections of botulinum toxin (totaling 0.8 LD.sub.50 units) were given over the scalp region. Surviving animals were subjected to autopsy and serial brain cutting and histologically stained using a standard Nissle formula. Serial cut mouse tissue sections were stained for Nissle substance using cresyl violet and immunostained for glutamate receptor activity. Sections were rinsed in TRIS-buffered saline with Tween 20 (TBS-T) containing 10% normal goat serum for one hour. Sections were then incubated overnight in TBS-T with 0.1% sodium azide and anti-GluR4. Sections were rinsed three times in TBS-T, followed by a 2-3 hour incubation in TBS-T containing a goat anti-mouse peroxidase-conjugated secondary antibody to detect glutamate. Sections were then rinsed three times in TBS-T. Antibody complexes were visualized using diaminobenzidine. Preabsorbtion with excess target protein, or omission of either primary or secondary antibody, were used to demonstrate antibody specificity and background generated from the detection assay. Tissue sections were examined using a Nikon Eclipse E800 microscope with a Spot RT digital camera. Photographs of tissue sections of neostriatum in an untreated mouse (sham injection) and a botulinum toxin treated mouse (four injections totaling 0.8 LD.sub.50 BOTOX® injected transdermally over the scalp reason) shown in FIG. 1.

### Example 17

To further understand whether to produce increased scientific evidence of the transcranial movement of botulinum toxin to brain parenchyma and brain structures, fusion between an Alexa dye and botulinum toxin has been used and placed into experimental rat model described herein. This fused preparation came from the same strain and culture used to manufactured version of Type A Toxin Hall strain typically used in treatment of human diseases as Botox (Trademark) distributed by Allergan Pharmaceuticals. This fused botulinum toxin Type A Neurotoxin with the Alexa dye was studied for possible diffusion into the brain from extra cranial injection at 3, 6, 12, 24 hours, and 2 weeks. A series of 3 to 5 mice were analyzed for each time period. At 6 hours the Alexa dye was noted in the brain and immunohistochemical analysis of central nervous system structures through the brain cuts and brain slices examined with immune fluorescent microscopy. At 3 hours no such dye was noted, and subsequent to that, no dye was noted. This was a pharmacokinetic experiment suggesting that the toxin and the dye together was moved into the brain from an extracranial injection.

This finding, combined with the anatomic atrophic changes at near lethal doses, changes in receptor expression for key brain neurotransmitters, indicates that there clearly is capability of botulinum toxin to move into the neuro cranium, and at least affect one neurotransmitter, have a biologic effect. Described herein is the utility from this experimental model that is helpful in the treatment of the diseases involving mood and affect, namely the manipulation of central nervous system neurotransmission involving neurotransmitters important to disorders of mood and affect as described herein.

### Example 18

An abnormality in glutamate neurotransmission is identified for a major depressive disorder within the basal ganglion and adjacent hemispheres compared to normal control comparative data. Symptoms of MDD are assessed according to criteria of the DSM IV. A treatment plan is constructed to apply a botulinum toxin in closer proximity to the rostral basal ganglion where the neurotransmitter abnormal activity is identified using anatomic parameters and calculating a dose dependent diffusion strategy,

Anatomic scans are over laid matching abnormal neurotransmitter activity (such as glutamate neurotransmission, glutamate content, or glutamate receptor expression) to anatomic regions so that the least distance is extrapolated from the neuro imaging studies. Varying doses of botulinum toxin are administered in a single or multiple dose location results in a targeted delivery into the central nervous system, brain from extra cranial injections.

### Example 19

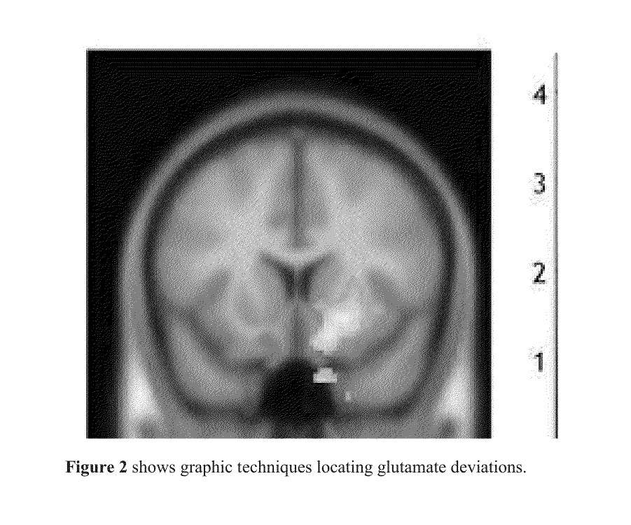

A 44 year old woman with major depression not responsive to serotonin reuptake inhibitor (Cymbalta™) and tricyclic anti-depressant and has been diagnosed as having at least 5 of the DSM IV determinant of major depression is analyzed with a PET scan set for glutamate neuro transmission and glutamate content with appropriate computer assisted program comparing against control levels in non depressed normal subjects, Deviation are measured by graphic techniques locating glutamate deviations in images such as given in FIG. 2.

Botulinum toxin type A is injected along the left periocular region, deep into mucosa of the left nasal cavity, and into the mid face. Botulinum toxin dosing is between 4-3000 unit combined however the dose is tailored based on nomogram determination of changes predetermined in multiple dosing test bin clinical studies with images which determine a signification biologic effect correlating region of the extra cranial anatomy, dose, number of injections, and effect on glutamate neurotransmission. The dose is applied in a fashion to mitigate adverse effect on muscles of facial expression so non facial muscular regions are preferred. Higher dose fractions are given into the muscle of face conducive to a cosmetic benefit so the facial asymmetry or disfigurement is avoided.

Assessment is made several weeks after for effect on major depression. Repeated injections are given at 6-14 week intervals. Various permeators such as hylauronidase or poly-cationic proteins may be used in injection strategy.

### Example 20

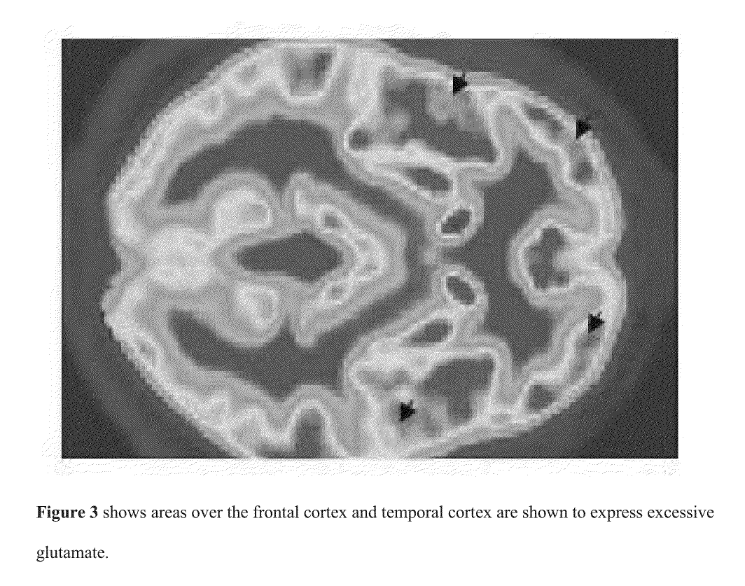

A 67 year old man is having deficits in memory and spatial orientation. He is clinically diagnostic as having Alzheimer's disease. The PET and Spectral MRI scan show increases in glutamate activity diffusely throughout the cerebral cortex but more prominent over frontal and temporal region. In order to limit neurotoxicity from glutamate in generation of the progression of Alzheimer's disease, multiple injections are given in the frontal and temporal region. In the FIG. 3, areas over the frontal cortex and temporal cortex are shown to express excessive glutamate. Botulinum is injected in bone structures over involved cerebral cortex causing a depression in glutamate activity and therefore a suppression of symptoms of organic brain dysfunction and mitigation in the progression of progressive dementia.

### Example 21

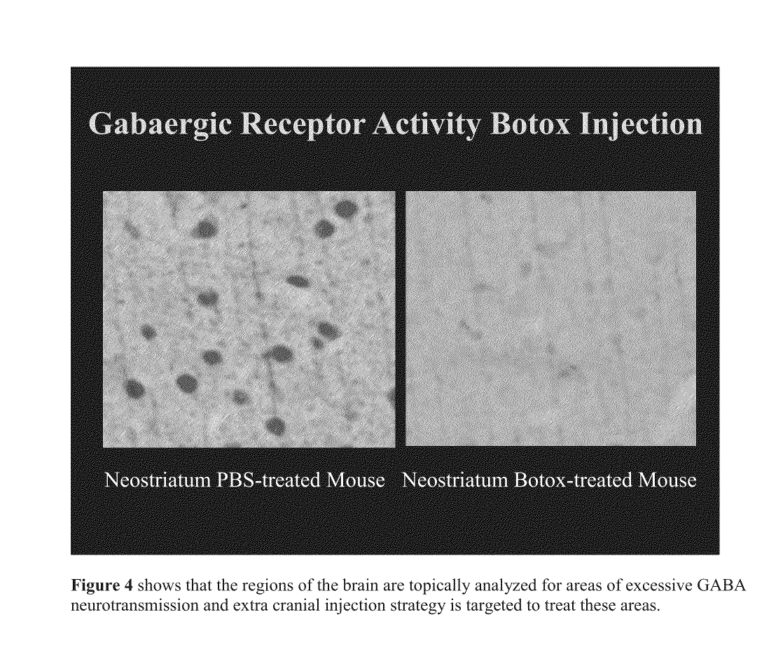

A patient is noted to have excessive GABA neurotransmission based on neuroimaging studies. FIG. 4 shows that the regions of the brain are topically analyzed for areas of excessive GABA neurotransmission and extra cranial injection strategy is targeted to treat these areas. Botulinum toxin is demonstrated to suppress GABA neurotransmission based on the animal studies demonstrated herein and an effective amount ranging between 5-3000 U is given in single or multiple locations in the extra cranial region.

I claim:

1. A method of treatment of a central nervous system disorder defined as an having an abnormality in at least one brain neurotransmitter in at least one or more regions of the brain comprising a method of:
a. injection of botulinum toxin into the extra cranial region of the head or neck
b. such that the extra cranial region of the head and neck is in close proximity with one region of the brain with abnormal neurotransmitter activity determination using a PET scan, functional Magnetic resonance imaging or brain scan
c. wherein the botulinum toxin blocks neurotransmission involving at least one neurotransmitter.

2. The method of claim 1, wherein the neurotransmitter is glutamate.

3. The method of claim 1, wherein the neurotransmitter is acetyl choline.

4. The method of claim 1, wherein the neurotransmitter is GABA.

5. The method of claim 1, wherein the neurotransmitter is nor epinephrine.

6. The method of claim 1, wherein the brain abnormality is associated with major depressive disorder.

7. The method of claim 1, wherein the brain abnormality is associated with bipolar disease.

8. The method of claim 1, wherein the brain abnormality is associated with general anxiety disorder and post-traumatic stress syndrome.

9. The method of 1, wherein the disorder is related to seizures.

10. The method of claim 9, wherein seizures include focal motor and partial complex.

11. The method of claim 1, wherein the disorder is a neuro degeneration characterized as Alzheimer's disease, Parkinson's disease, or Huntington's disease.

12. The method of claim 1, wherein is a disorder is cerebral 1 vascular accident.

13. The method of claim 1, wherein is central mediated hypertension.

14. The method of claim 13, wherein is the cocentral component of essential hypertension.

15. The method of claim 1, wherein is an appetite disorder which is obsessive compulsive disorder.

16. The method of claim 1, wherein is a circadian rhythm disorder.

17. The method of claim 1, wherein is a sleep disorder.

18. The method of claim 1, wherein is a pain disorder characterized as migraine, tension headache, combine tension headache migraine, trigeminal neuralgia, myofascial pain, cluster headache, post operative wound pain.

19. The method of claim 1, wherein is schizophrenia.

20. The method of claim 1, wherein is a seasonal affective disorder.
